(19) United States
(12) Patent Application Publication
(10) Pub. No.: US 2010/0055142 A1
(43) Pub. Date: Mar. 4, 2010

(54) Carrier Neutralization/Modification in Antimicrobial Compositions, Articles and Methods

(75) Inventors: David G. Heagle, Taunton, MA (US); Chirag B. Shah, North Attleboro, MA (US); Ronald F. Vitaris, Worcester, MA (US); E. David Fink, Franklin, MA (US); Sharon A. Mulligan, Bristol, RI (US); Brian Dowd, Dedham, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/548,514

(22) Filed: Aug. 27, 2009

Related U.S. Application Data
(60) Provisional application No. 61/136,324, filed on Aug. 28, 2008.
(60) Provisional application No. 61/140,020, filed on Dec. 22, 2008.

(51) Int. Cl. A01N 25/34; B05D 3/00; A61K 47/48; A01N 25/26; A61F 13/00; A01N 25/04

(52) U.S. Cl. 424/404; 427/2.3; 424/78.17; 424/490; 424/443; 424/484

(57) ABSTRACT

A method of treating a substrate having a charge bias with at least one antimicrobial agent to modify the release properties of the antimicrobial agent with respect to the substrate, the method includes eliminating, mitigating, or modifying the charge bias of the substrate by applying at least one first agent to the substrate, and applying the at least one antimicrobial agent to the substrate. Related articles are also described.

### FIELD

The present invention is directed to antimicrobial compositions, articles and methods.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

A variety of anti-microbial compositions, articles and methods have been suggested. However, such wound compositions and methods possess various deficiencies and shortcomings.

Some antimicrobial agents, such as polymeric biguanides, exhibit antimicrobial activity via their multiple positively charged biguanide functional groups. This polycationic structure attracts and attaches to negatively charged microbial membrane surface. During manufacture or clinical use, the polymeric biguanide containing dressing may come into contact with anionic systems that may partially or fully inactivate the polymeric biguanide.

For example, the antimicrobial effectiveness of some antimicrobial compounds (e.g., PHMB) applied to a carrier or substrate (e.g., a cellulosic such as cotton) is strongly influenced by interaction of the positive charge of the antimicrobial agent and the negative charge of the carrier or substrate. For example, an antimicrobial such as PHMB is a polycationic compound with multiple positive charges on one molecule. A carrier or substrate (e.g., a cellulosic such as cotton) can have multiple negative charges, especially near a pH of 7. The interaction of the multiple positive charges associated with the antimicrobial agent (e.g., PHMB) with multiple negative charges of the carrier or substrate (e.g., cotton) contributes to a very strong bonding of the antimicrobial to the carrier or substrate. When the antimicrobial compound is strongly bonded to the carrier or substrate it is not readily released therefrom. In those instances where a readily releasable antimicrobial agent is desired, this strong bonding of the antimicrobial agent to the carrier or substrate can be disadvantageous. Additionally, when the positive charges interact with negative charges, the charges are neutralized and thus are not available to provide optimal antimicrobial efficacy. Moreover, the negative charges on the substrate may not generally be of the ideal nature or strength to achieve optimal bonding and/or release of an antimicrobial agent therefrom.

The present invention may optionally addresses one or more of the above-mentioned problems/deficiencies associated with conventional antimicrobial compositions, articles, and methods.

### DEFINITIONS

As used herein, unless otherwise indicated, the terms “microbial organism” or “microbial” will be used to refer to microscopic organisms of matter, including fungal, bacterial and/or viral organisms. Thus, the term “antimicrobial” as used herein refers to a composition or agent that kills or otherwise inhibits the growth of such fungal, bacterial and/or viral organisms.

### SUMMARY

Thus, the present invention may optionally provide compositions, articles and methods which address one or more of the above mentioned shortcomings associated with the relevant conventional technologies. Therefore, the present invention may optionally provide compositions, articles, systems and/or methods that would limit exposure of polymeric biguanide molecules, or similar antimicrobial agents, to incompatible compounds other than undesirable microbes.

The present invention may optionally possess one or more of the following features, benefits or advantages: highly active compositions or articles such as a wound dressing with a relatively reduced concentration of antimicrobial agents such as PHMB and/or PEHMB, thereby enhancing clinical safety; increased wear time of an antimicrobial dressing that can wick and hold fluid away from the wound site while decreasing the chances of bacterial growth within the dressing, thereby permitting less frequent changes of the dressing and therefore more efficient and economical and care management.

The present invention, according to certain aspects, involves neutralization or modification of charge bias, which may be present on a carrier or substrate for one or more antimicrobial substance(s).

According to certain embodiments of the present invention, neutralization or modification of charge bias on the carrier or substrate material can provide a way to optimize available binding sites for antimicrobial agent(s) and a method to control attachments, release profiles, as well as antimicrobial activity. Accordingly, the substrate may be treated to provide a less aggressive bonding to an antimicrobial agent applied thereto. Thus, the antimicrobial agent is more easily released therefrom. In addition, the charge bias present in the antimicrobial agent or composition is not negated by the carrier or substrate material, thereby providing a more effective antimicrobial activity.

According to certain embodiments, the present invention can use one or more of an inorganic and an organic cationic compound to neutralize negative charge bias on a carrier or substrate material prior to, concurrently with or subsequent to, application of an antimicrobial agent thereto.

According to a first aspect, the present invention provides a method of treating a substrate having a charge bias with at least one agent to modify the release properties of the antimicrobial agent with respect to the substrate, the method comprising: (a) eliminating, mitigating, or modifying the charge bias of the substrate by applying at least one first agent to the substrate; and (b) applying the at least one antimicrobial agent to the substrate.

According to another aspect, the present invention provides an article comprising a substrate, the substrate comprising a surface, at least a portion of the surface having a charge bias eliminated, mitigated or modified by at least one first agent, the article further comprising at least one antimicrobial agent releasable from the portion of the surface.

According to yet another aspect, the present invention provides a wound dressing made according to the methods described above, or formed from an article of the type described above.

### BRIEF DESCRIPTION OF THE DRAWINGS

FIG. 1 is a sectional view of a laminate or dressing formed according to the principles of the present invention.

### DETAILED DESCRIPTION

According to certain embodiments, the present invention comprises a wound dressing containing an antimicrobial agent, such as polymeric biguanides (e.g., polyhexamethylene biguanide (PHMB) and/or polyethylene hexamethylene biguanide (PEHMB)), as well as means to prevent or mitigate inactivation of the antimicrobial agent during manufacture and/or use.

According to its broader aspects, the present invention is directed to an article comprising an antimicrobially treated carrier or substrate, Preferably, one or more of the carrier or substrate, and the antimicrobial agent, have been constructed, formulated or treated in a manner which eliminates, mitigates or modifies charge bias present on the carrier or substrate which may have the above-mentioned adverse impacts on the antimicrobial performance of the article. The present invention is also directed to methods or techniques for eliminating, mitigating or modifying the above-mentioned charge bias in an antimicrobially treated article.

Any suitable antimicrobial agent can be utilized. According to certain nonlimiting examples, polymeric biguanides such as PHMB, PEHMB, or derivatives thereof can be utilized as the antimicrobial agent(s). Alternatively, certain metals, or compounds including such metals, such as silver, gold, copper or zinc may be used as the antimicrobial agent(s). It is additionally contemplated that the antimicrobial treatment could be a combination of a number of agents such as silver, PHMB, CHG, EDTA or other suitable antimicrobials such that a synergistic efficacy is realized. It should be noted that when two or more polycationic antimicrobial agents are utilized (e.g., chitosan and PHMB), these agents compete for any negative charges present on the carrier or substrate, thus amplifying the problems described herein to which the present invention may be directed. It should be noted that when two or more cationic compounds are utilized with different molecular weights and charge density (i.e., a combination of lower and higher charge density or molecular weight) these two or more compounds can be added simultaneously or in two steps, and differential adsorption can occur on the carrier or substrate.

According to certain embodiments, the antimicrobial agent(s) can comprise a cationic surfactant or a cationic quaternary ammonium compound. Non-limiting examples of such compounds include: benzalkonium chloride; benzethonium chloride; cetrimide; cetylpyridinium chloride; chlorphenoctium amsonate; dequalinium acetate; dequalinimum chloride; domiphen bromide; laurolinium acetate; methylbezethonim chloride; myristyl-gamma-picolinium chloride; ortaphnum chloride; triclobisonum chloride; cetalkonium chloride; dofanium chloride; tetraethylammonum bromide; didecyldimethylammonium chloride; tetraethylammonium bromide; dimethyldiallyl ammonium chloride; p-trialkylamioethyl styrene monomer; and trialkyl(p-vinylbenzyl) ammonium chloride.

According to further embodiments, the antimicrobial agent(s) can comprise a cationic surfactant or a polymeric quaternary ammonium compound. Non-limiting examples of such compounds include: poly(diallyl dimethyl ammonium chloride); poly(3-chloro-2 hydroxypropyl) methacryloxyethyl dimethyl-ammonium chloride; poly(acrylamide-methacryloxyethyl trimethyl-ammonium bromide; poly(butyl acrylate-methacryloxyethyl trimethylammonium bromide; poly(1-methyl-4-vinyl pyridinium bromide); poly(1-methyl-2-vinylpyridinium bromide); and poly(methylacryloxyethyl triethyl ammonium bromide).

According to additional alternative embodiments, the antimicrobial agent(s) can comprise a polyquaternium. Polyquaternium is a neologism used to emphasize the presence of quaternary ammonium centers in the polymer. Polyquaterniums are positively charged, and some have antimicrobial properties. There are currently at least 37 different known polymers under the polyquaternium designation. New polyquanterniums are identified periodically. Different polymers are distinguished by the numerical value that follows the word “polyquaternium.” Thus, the present invention contemplates the possible use of any of the currently known polyquaternium-1 through polyquaternium-37 substances, as well as future polyquanterniums, currently undesignated, falling under the broad definition or categorization noted above.

According to further embodiments, the antimicrobial agent(s) can comprise a cationic antimicrobial peptide, such as e-poly-l-lysine, magainin, cecropins, dermaseptin, pexiganan, iseganan, Oniganan, and defensin.

According to additional alternatives, the antimicrobial agent(s) can comprise amphoteric surfactants, such as include alkyl betaines, dodecyl betaine cocoampho glycinate, and cocamidopropyl betaine.

According to further embodiments, the antimicrobial agent(s) can comprise bromine based compounds such as poly(4-vinyl-N-alkyl pyridinium bromide); and poly(4-vinyl-N-hexylpyridinium bromide).

The carrier or substrate can take any suitable form. The carrier or substrate can comprise particles, beads, spheres, flat sheets (continuous or discrete), rolls, foam, and three-dimensional shapes and configurations.

The carrier or substrate can be composed of a dispersed particle system including aerosols, emulsions, hydrosols, organosols, slips, slurries, sols, and suspensions. These particle systems can be heterodispersed, polydispersed or monodispersed. The particle size can vary from colloidal to course granules. For example, the particle size can vary from about 10 Å to about 10,000 Å for colloidal particles, to about 50 μm to about 5 mm for coarse particles or granules. The structure of these dispersed particle systems can include droplets, microspheres, aggregates, agglomerates, coagulates, flocs, powders, gels, aerogels, alcogels, hydrogels and xerogel. These particle systems can be associated based on aggregation, agglomeration, coagulation, flocculation, gelation, fusion or sol-gelation. Additionally these particle systems can be disassociated based on deagglomeration, defloccuation, comminution, or peptization. The stability of these systems may be characterized as colloidal, kinetic, stable or unstable. The stability of these systems may be controlled by electrostatic, steric, electrosteric or depletion mechanisms

The carrier or substrate can be manufactured from a variety of fibers which may include natural fibers, synthetic fibers, or combinations thereof. Thus, suitable fibers can be formed from metal, ceramics, polymers, or natural materials. Non-limiting examples include: cotton, cellulose, polyester, polyethylene, polypropylene, PTFE, nylon, aramids, Kevlar, chitosan, alginates, poly(ethylene terephthate) (PET), acrylics, fluorocarbons, modacrylics, polyesters, rubber, saran, spandex, vinal, vinyon, rayon, acetate, triacetate, protein, flax, hemp, jute, ramie, manila, kapok, wool, or silk.

The fiber can have any suitable size, such as an effective diameter from 5 nm to 5 mm and the specific surface area can vary from 0.001 to 1000 m²/g. The cross section of the fibers can be delta, circular, fibrillated, or 4DG™ (commercially available from Fiber Innovation Technology, Inc., Johnson City, Tenn.; see also, Heather L. Paul et al., “Comparison of Thermal Insulation Performance of Fibrous Materials for the Advanced Space Suit,” Journal of Biomechanical Engineering, Volume 125, October 2003, Pages 639-647; entire contents incorporated herein by reference); or any other suitable shape.

Fibers can be combined in any suitable fashion, such as woven, non-woven, knit, felt, or braided. The fibers can be continuous fiber or tow, cut staple fiber, wet laid/paper, meltblown, flash spun fibrillated tape, spunbond, needle punched, carded, composite structures, thermal bonded, chemical bonded, hydroentangled, airlaid, drylaid, highloft, ultrasonically bonded, stitchbonded, or powderbonded.

The carrier or substrate could be a foam. This foam could be composed of polyurethane, olefin, PVC, polypropylene, polyethylene, EVA, ESI, or other polymers. The foam could be a bead gas formed foam or a foam formed by any other suitable process. The foam could be open or closed cell, with 5 to 200 pores per inch (ppi). A closed cell foam could be formed by thermal, caustic or other means of reticulation. The density of the foam could vary from 1 to 5 lb/ft³.

The carrier or substrate could also be a film. This film could be composed of many synthetic, manmade or natural polymers. The film could be perforated or fibrillated.

According to another optional aspect of the present invention, a carrier or substrate is treated with one or more neutralizing or enhancement agent(s) prior to, or concurrently with, application of an antimicrobial agent thereto. According to one optional embodiment, the one more neutralizing or enhancement agents also possess an antimicrobial effect. According to certain nonlimiting examples, the carrier or substrate is treated with an inorganic and/or organic neutralizing or enhancement agent(s). Any suitable inorganic or organic substance(s) may be utilized. For example, alum, aluminum ammonium sulfate, and/or polyethyleneimine can be utilized. According to one specific nonlimiting example, a cellulosic substrate, such as cotton is treated with both an inorganic and organic compound, such as the compounds described above prior to application of an antimicrobial agent (e.g., PHMB).

A number of different suitable neutralizing or enhancement agents are contemplated by the present invention. As noted above, the neutralizing agent can be inorganic. Suitable inorganic neutralizing agents include: Al₂(SO₄)₃.14 to 18H₂O; and AlCl₃.6H₂O Fe₂(SO₄)₃.9H₂O, FeCl₃ Na₂Al₂O₄. Other suitable agents include soluble salts liberating mono or multivalent cations such as Ag+, Ca++, Mg++, Zn++, etc. In another optional aspect of the invention, another positively charged compound can be attached to the dressing. Examples include chitosan and quaternary ammonium compounds such as Benzanlkonium chloride.

The neutralizing or enhancement agent could be a zwitterionic compound. Non-limiting examples of such compounds include: amino acid; amino-sulfonic acid based 2-(N-morpholino)ethanesulfonic acid (MES); 3-(N-morpholino)propanesulfonic acid (MOPS); 4-(2-hydroxyethyl)-1-piperazineethanesulfonic acid (HEPES); piperazine-N,N′-bis(2-ethanesulfonic acid) (PIPES); N-cyclohexyl-3-aminopropanesulfonic acid (CAPS); amino-carboxylic acid (amino acid) based glycine, its derivatives bicine and tricine; alanine; and combinations thereof.

The neutralizing or enhancement agent could comprise a polyelectrolyte in the form of a fiber (e.g., ultra-fine fibers); a hydrogel (e.g., acrylic acid polyelectrolyte hydrogel); a network (e.g., block polyelectrolyte networks containing cross-linked poly(acrylic acid) (PAA) and poly(ethylene oxide) (PEO)). Regardless of its form, suitable polyelectrolytes include: poly(diallyldimethylammonium chloride); poly(allylamine hydrochloride); diallyldimethylammonium chloride; poly(acrylamide-co-diallyldimethylammonium chloride); and combinations thereof.

The neutralizing or enhancement agent could comprise a quaternized hydroxyethyl cellulose (HEC) polymer (e.g., as commercially available from Amerchol as SoftCAT™ family of products)

The neutralizing or enhancement agent could comprise a cationic cellulosic polymer (e.g., as commercially available from National Starch as CELQUAT® L-200).

The neutralizing or enhancement agent could comprise highly charged cationic copolymers of diallyl dimethyl ammonium chloride and acrylic acid (e.g., as commercially available from Nalco as MERQUAT® series of products)

The neutralizing or enhancement compound could alternatively comprise a natural, semisynthetic or synthetic a cationic polysaccaride. Non limiting examples include chitosan, hydroxyethyl cellulose, guar gum, and hydroproyl guar. The neutralizing or enhancement compound could alternatively comprise amphoteric polysaccharide. Non limiting examples include carboxymethylated chitosan and modified potato starch.

Additional negative charge could be induced with an additional neutralizing or enhancement agent, such as carboxymethylcellulose (CMC), cyclodexdrin, poly(sodium styrene sulfonate) (PSS), poly L-Glutamate; and combinations thereof.

According to one optional aspect of the present invention, the carrier or substrate is treated with a neutralizing or enhancement agent comprising polyampholytes, which are charged polymers with both positively and negatively charged groups.

According to further optional embodiments, the neutralizing or enhancement agent can comprise a first fraction capable of penetrating below an outer surface of the substrate, and a second fraction interacting with an outer surface portion of the substrate. For example, the neutralizing or enhancement agent may comprise a cationic polyelectrolyte, the first fraction comprising a low molecular weight fraction of the polyelectrolyte, and the second fraction comprising a high molecular weight fraction of the polyelectrolyte. Only the first fraction is able to penetrate below an outer surface portion of the substrate to interact with a charge bias present within the substrate below an outer surface portion thereof. The interaction with the substrate of the second fraction would be limited to an outer surface portion, since the high molecular weight fraction would be unable to penetrate into the substrate.

According to one optional aspect of the present invention, the carrier or substrate is treated so as to eliminate, mitigate or reduce charges which may lie below the surface. Thus, the antimicrobial agent would attach mostly to charges present on the surface of the carrier or substrate only.

The carrier or substrate may be treated in stages. In a first stage, the charge neutralization or enhancement agent of the type described herein can be applied to the substrate by a variety process including padding, spraying, gravure roll, slot coating, etc., followed by an optional drying step. The charge neutralization or enhancement agent can optionally be applied in the form of a solution in the first stage, and the solution can be provided with a pH to optimize the treatment. One or more surfactant(s) may also optionally be used in the first stage of the treatment. In a second stage, the dried carrier or substrate produced by the first stage is treated with an antimicrobial agent of the type described herein by a variety processes including padding, spraying, gravure roll, slot coating etc., followed by an optional drying step and optional second application of an antimicrobial or other therapeutic agent. The antimicrobial agent(s) may optionally be applied in the form of a solution in the second stage. The pH of the solution can varied or chosen to optimize the treatment. One or more surfactant may also be used in the second treatment phase. A drying step may optionally follow the second phase of treatment. The drying temperature may be varied to optimize the performance of the antimicrobial agent(s).

According to an alternative embodiment, the different stages described above can be merged into a single treatment phase, For example, the substrate can be treated with a combination of neutralizing or enhancement agent(s) and antimicrobial agent(s). This combination may optionally be applied to the substrate in the form of a solution, with a pH optionally selected to optimize the treatment of the substrate. An optional drying step may also be performed, as set forth above.

As a further additional alternative modification of the techniques described herein, only a potion of a surface on the substrate or carrier surface need to be exposed to the neutralization or enhancement agent(s), and/or the antimicrobial agent(s). Thus, for example, the substrate may be folded or stretched thereby exposing or hiding selective areas of one or more surfaces present on the substrate for exposure to the above-described treatment. Alternatively, masking techniques can be utilized to shield certain areas of at least one surface of the substrate or carrier from the treatment. Any suitable masking technique can be utilized, such as those currently utilized in silicon chip preparation and manufacture. According to a further optional modification, the antimicrobial agent(s) may be applied to those portions on a surface of the substrate which were shielded from exposure to the neutralization or enhancement agent. According to yet another modification, the antimicrobial agent can be applied to both shielded and exposed portions on the surface of the substrate. Utilizing these techniques it can be seen that the antimicrobial release signature of a treated substrate can be tailored to suit a particular need. For example, a central area of the substrate can be exposed and treated with the above-mentioned neutralization agent, while a surrounding peripheral portion is shielded from exposure thereto. An antimicrobial agent is then applied to the entire substrate. In the instance where the charge of bias of the substrate has been neutralized, the antimicrobial agent will be more loosely bound to the central area of the substrate, and more tightly bound to the surrounding peripheral portion. Thus, if the substrate is utilized in the form of a wound dressing, the central portion can be placed over the wound, such that the antimicrobial agent is more freely released to treat the wound, while the antimicrobial agent is more tightly bound in the surrounding peripheral area to kill pathogens within the dressing as they attempt to enter the wound site.

Whether the antimicrobial agent is more tightly or more loosely bound to the substrate as a result of the treatment depends on the type of charge bias modification imparted by the neutralization or modification agent. For example, if the substrate possesses a negative charge bias, and the neutralization or modification agent is cationic, the charge neutralization agent binds or occupies charges present on the carrier or substrate, and the antimicrobial or therapeutic agent applied in the second phase will be more releasably bound thereto. In other words, the antimicrobial agent will be more freely released from the substrate.

Thus, according to the principles of the present invention, a wound dressing can be designed and constructed having multiple functionality or antimicrobial release signatures. Depending on the course of treatments and modification of the charge biases at different thicknesses within the carrier or substrate, a wound dressing formed from a single layer carrier or substrate material can be provided which has different substances embedded therein throughout the thickness thereof, in which substances can either bind tightly to the carrier or substrate, or which may be more readily released thereby. Thus, a single layer wound dressing can be produced which provides the functionality similar to that of a multilayer wound dressing. According to further optional embodiments of the present invention, two or more substrate materials can be separately treated with charge-bias modifying compounds, such as polycationic or polyanionic agents. Subsequent to treatment, these different substrate materials can be woven together, or layered to form a customized wound dressing material.

According to yet another optional embodiment of the present invention, suitable carrier or substrate (e.g., cotton) can be surface treated not only to neutralize negative charges present there on, but to also add certain functional groups to the carrier or substrate that could bind to groups of a suitable antimicrobial agent (e.g. PHMB), thereby leaving positive charges associated with the antimicrobial agent more available for carrying out its antimicrobial effect. Those skilled in the art are familiar with a number of suitable techniques for applying such functional groups. According to nonlimiting examples, the carrier or substrate may be plasma treated or chemically treated to associate the above-mentioned functional groups therewith. Any suitable functional group may be utilized for this purpose. According to one aspect of the present invention, the attached functional groups have two end functional groups; one end constructed to react or bind with the carrier or substrate, and the second end constructed to react or bind with the antimicrobial agent(s).

The above-mentioned functional groups, as well as a variety of other constituents with polycationic and polyanionic charges, can be added to an article, such as a wound dressing by known electrostatic layer-by-layer self-assembly techniques.

In another aspect of the invention, the polymeric biguanide molecule on the dressing may be complexed with negatively charged compounds. It is beneficial to have only ionic interaction between the polymeric biguanide and anionic compounds. In presence of wound fluid this ionic interaction may be broken in favor of stronger attraction toward a microbial membrane surface. Glycosaminoglycans are one example of a group of such compounds that may only ionically interact with the polymeric biguanide. Another example may be a cell signaling molecule, material or coating such that the cell-signaling molecule exhibits a greater affinity or attraction to the antimicrobial agent than other cations. Those signaling molecules could also detect a change in bacterial phenotype or virulence such that the agent would respond to a more pathogenic response from the cell and activate an antimicrobial activity.

In another aspect of the invention, an electric field may be applied to the dressing to uncouple cations or separate cationic materials from anionic materials.

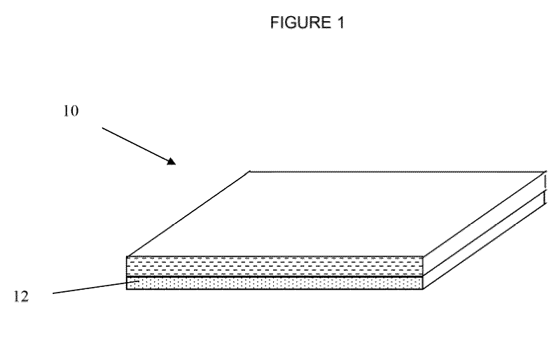

As illustrated in FIG. 1, in an optional alternative form of the present invention, the dressing 10 may be configured to exclude absorbance of wound fluid components based on size exclusion principles. One example is attachment of semi permeable film 12 on one side of the dressing 10 that would be exposed to wound fluid. The film 12 could optionally comprise an array of apertures which vary in pattern, number and opening diameter to help regulate fluid movement. The apertures could be constructed such that they promote flow in only one direction using simple valves, flaps or like technologies.

Wound dressings can, of course, include additional active ingredients or agents such as, for example, a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to induce directional bacterial growth, an agent to impart bactericidal or bacteriostatic activity, an electron transfer agent to destabilize or destroy the metabolic action of microbes and/or biofilm formation, combinations thereof and the like. Release of active agents may be triggered by a variety of means, such as, for example, an electric field or signal, temperature, time, pressure, moisture, light (e.g., ultra-violet light), ultrasound energy, sonication, combinations thereof and the like.

Any numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112, ¶16, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention.

We claim:

1. A method of treating a substrate having a charge bias with at least one antimicrobial agent to modify the release properties of the antimicrobial agent with respect to the substrate, the method comprising:
(a) eliminating, mitigating, or modifying the charge bias of the substrate by applying at least one first agent to the substrate; and
(b) applying the at least one antimicrobial agent to the substrate.

2. The method of claim 1, wherein (a) and (b) are performed sequentially.

3. The method of claim 1, wherein (a) and (b) are performed simultaneously.

4. The method of claim 1, further comprising: drying the substrate.

5. The method of claim 4, wherein the drying is performed subsequent to (a).

6. The method of claim 4, wherein the drying is performed subsequent to (b).

7. The method of claim 4, wherein the drying is performed subsequent to (a), and performed again subsequent to (b).

8. The method of claim 6, wherein no further treatment steps are performed subsequent to the drying.

9. The method of claim 1, wherein the charge bias is anionic, and wherein the at least one first agent is cationic.

10. The method of claim 1, wherein (a) and (b) are performed by at least one of: padding, spraying, gravure roll, and slot coating.

11. The method of claim 1, wherein the at least one first agent and the at least one antimicrobial agent are applied to the substrate in the form of a solution.

12. The method of claim 1, further comprising shielding at least a portion of the substrate from exposure to the at least one first agent during (a), and exposing the shielded portion of the substrate during (b).

13. The method of claim 1, further comprising surface treating the substrate to associate one or more functional groups therewith.

14. The method of claim 13, wherein the functional groups comprise both anionic and cationic groups.

15. The method of claim 13, comprising surface treating the substrate with a polyampholyte.

16. The method of claim 13, wherein the surface treating comprises at least one of: wet chemical reaction; organosilanization; ionized gas treatments; UV radiation; and tethering with an intermediary.

17. The method of claim 1, wherein the at least one first agent comprises a first fraction capable of penetrating below an outer surface of the substrate, and a second fraction interacting with an outer surface portion of the substrate.

18. The method of claim 17, wherein the at least one first agent comprises a cationic polyelectrolyte, the first fraction comprises a low molecular weight fraction of the polyelectrolyte, and the second fraction comprises a high molecular weight fraction of the polyelectrolyte.

19. The method of claim 1, wherein the substrate comprises: particles, beads, spheres, continuous sheets, discrete sheets, foams, gels or 3-dimensional shapes.

20. The method of claim 1, wherein the substrate is formed from a material comprising: glass, ceramic, metal or polymer.

21. The method of claim 1, wherein the substrate comprises: natural fibers; synthetic fibers; or combinations thereof.

22. The method of claim 21, wherein the fibers are formed from: cotton, cellulose, polyester, polyethylene, polypropylene, PTFE, nylon, aramid, Kevlar, chitosan, alginate, poly(ethylene terephthate), glass, ceramics, metal, acrylics, fluorocarbon, modacrylic, polyester, rubber, saran, spandex, vinal, vinyon, rayon, acetate, triacetate, protein, flax, hemp, jute, ramie, manila, kapok, wool, silk; or combinations thereof.

23. The method of claim 21, wherein the fibers are: woven; non-woven; knit; felt; braided; continuous fiber or tow; cut staple fiber; wet laid/paper; meltblown; flash spun fibrillated tape; spunbond; needle-punched: carded; composite structures; thermal bonded; chemically bonded; hydroentangled; airlaid; drylaid; highloft; ultrasonically bonded stitchbonded powderbonded; or combinations thereof.

24. The method of claim 1, wherein the at least one first agent comprises: alum; aluminum ammonium sulfate; polyethyleneimine; or combinations thereof.

25. The method of claim 1, wherein the at least one first agent comprises: a zwitterionic compound; a polyelectrolyte; a quaternized hydroxyethyl cellulose polymer; a combination of cationic copolymers; a cationic polysaccaride; carboxymethylcellulose; or combinations thereof.

26. The method of claim 1, wherein the at least one antimicrobial agent comprises: a polymeric biguanide; a cationic quaternary ammonium compound, a polymeric quaternary ammonium compound; a polyquaternium; a cationic antimicrobial peptide; or combinations thereof.

27. An article comprising a substrate, the substrate comprising a surface, at least a portion of the surface having a charge bias eliminated, mitigated or modified by at least one first agent, the article further comprising at least one antimicrobial agent releasable from the portion of the surface.

28. The article of claim 27, wherein the charge bias is anionic, and wherein the at least one first agent is cationic.

29. The article of claim 27, wherein at least a portion of the surface comprises one or more functional groups associated therewith.

30. The article of claim 29, wherein the functional groups comprise both anionic and cationic groups.

31. The article of claim 27, wherein the at least one first agent comprises a first fraction capable of penetrating below an outer portion of the surface, and a second fraction interacting with the outer portion of the surface.

32. The article of claim 31, wherein the at least one first agent comprises a cationic polyelectrolyte, the first fraction comprises a low molecular weight fraction of the polyelectrolyte, and the second fraction comprises a high molecular weight fraction of the polyelectrolyte.

33. The article of claim 27, wherein the substrate comprises: particles, beads, spheres, continuous sheets, discrete sheets, foams, gels or 3-dimensional shapes.

34. The article of claim 27, wherein the substrate is formed from a material comprising: glass, ceramic, metal or polymer.

35. The article of claim 27, wherein the substrate comprises: natural fibers; synthetic fibers; or combinations thereof.

36. The article of claim 35, wherein the fibers are formed from: cotton, cellulose, polyester, polyethylene, polypropylene, PTFE, nylon, aramid, Kevlar, chitosan, alginate, poly(ethylene terephthate), glass, ceramics, metal, acrylics, fluorocarbon, modacrylic, polyester, rubber, Saran, spandex, vinal, vinyon, rayon, acetate, triacetate, protein, flax, hemp, jute, ramie, manila, kapok, wool, silk; or combinations thereof.

37. The article of claim 35, wherein the fibers are: woven; non-woven; knit; felt; braided; continuous fiber or tow; cut staple fiber; wet laid/paper; meltblown; flash spun fibrillated tape; spunbond; needle-punched; carded; composite structures; thermal bonded; chemically bonded; hydroentangled; airlaid; drylaid; highloft; ultrasonically bonded stitchbonded powderbonded; or combinations thereof.

38. The article of claim 27, wherein the at least one first agent comprises: alum; aluminum ammonium sulfate; polyethyleneimine; or combinations thereof.

39. The article of claim 27, wherein the at least one first agent comprises: a zwitterionic compound; a polyelectrolyte; a quaternized hydroxyethyl cellulose polymer; a combination of cationic copolymers; a cationic polysaccaride; carboxymethylcellulose; or combinations thereof.

40. The article of claim 27, wherein the at least one antimicrobial agent comprises: a polymeric biguanide; a cationic quaternary ammonium compound, a polymeric quaternary ammonium compound; a polyquaternium; a cationic antimicrobial peptide; or combinations thereof.

41. A wound dressing formed from the article of claim 27.
